(19) United States
(12) Patent Application Publication
(10) Pub. No.: US 2026/0308099 A1
(43) Pub. Date: Oct. 8, 2026

(54) STORAGE AND/OR TRANSPORT OF ETHYLENICALLY UNSATURATED CARBOXYLIC ACIDS

(71) Applicant: BASF SE, Ludwigshafen am Rhein (DE)

(72) Inventors: Jan Niclas GORGES, Ludwigshafen am Rhein (DE); Tile GIESHOFF, Ludwigshafen am Rhein (DE); Peter ZUROWSKI, Ludwigshafen am Rhein (DE); Friederike FLEISCHHAKER, Ludwigshafen am Rhein (DE); Rebecca SURE, Ludwigshafen am Rhein (DE); Christian REIN, Ludwigshafen am Rhein (DE); Friedrich-Georg MARTIN, Ludwigshafen am Rhein (DE)

(21) Appl. No.: 18/874,613

(22) PCT Filed: Jun. 6, 2023
(86) PCT No.: PCT/EP2023/065092

§ 371 Date: Dec. 13, 2024

(30) Foreign Application Priority Data
Jun. 17, 2022 (EP) 22179523.0

(51) Int. Cl. C07C 51/50; C07C 57/04; C08F 2/38

(52) U.S. Cl. CPC C07C 51/50; C07C 57/04; C08F 2/38

(57) ABSTRACT

The present invention relates to a method for storing and/or transporting an ethylenically unsaturated carboxylic acid, the ethylenically unsaturated carboxylic acid being protected with manganese(II) ions against undesired radical polymerization.

The present invention relates to a method of storage and/or transport of an ethylenically unsaturated carboxylic acid, wherein the ethylenically unsaturated carboxylic acid is protected against undesired free-radical polymerization with manganese(II) ions.

Chemical compounds having one or more ethylenically unsaturated groups have a pronounced tendency to free-radical polymerization. Such compounds are therefore subsequently also referred to as polymerizable compounds. The tendency of these compounds to free-radical polymerization means that they are used as monomers for the production of polymers. However, the pronounced tendency to free-radical polymerization of these compounds is also a disadvantage in that undesired spontaneous free-radical polymerization can occur during storage and transport and also during chemical and/or physical processing such as distillation or rectification, particularly under the action of energy such as heat and/or light. Such uncontrolled polymerizations can result in the gradual formation of polymer deposits, for example on heated surfaces, that necessitates removal of the polymer deposits and thus often results in shortened operating times. The uncontrolled polymerizations may even proceed explosively.

During storage and transport and also during chemical and/or physical processing of ethylenically unsaturated compounds that have a tendency to free-radical polymerization or mixtures that comprise such compounds, it is therefore customary to add compounds that prevent or at least retard undesired spontaneous free-radical polymerization. Such substances are referred to as polymerization inhibitors.

Polymerization inhibitors may be employed as individual chemical compounds or as mixtures of compounds. Particular requirements are placed on the polymerization inhibitor, depending on its field of use. For a polymerization inhibitor to be suitable as a transport and/or storage stabilizer of ethylenically unsaturated compounds it is important that the efficiency of the polymerization inhibitor, i.e. the extent of inhibition of polymerization, is controllable. Under the conditions of storage and/or transport of the ethylenically unsaturated compounds, the polymerization inhibitor should adequately prevent or retard the undesired spontaneous free-radical polymerization, whereas the desired free-radical polymerization of the ethylenically unsaturated compounds should be possible under appropriate polymerization conditions without the need to first have to separate the polymerization inhibitor used during storage and/or transport. If the stabilizer used during storage and/or transport is not separated for the desired polymerization, it is important that it does not adversely affect the desired polymerization, for example does not unintentionally act as a chain-transfer agent.

In terms of global production volume, acrylic acid is without doubt one of the most important ethylenically unsaturated compounds. During storage and/or transport, acrylic acid is normally stabilized against undesired spontaneous free-radical polymerization with 0.018% to 0.022% by weight of hydroquinone monomethyl ether (MEHQ), based on the amount of acrylic acid. For adequate stabilization of acrylic acid against undesired spontaneous free-radical polymerization using MEHQ, it is necessary that oxygen is dissolved in the acrylic acid in sufficient amounts. Sufficient amounts of oxygen are generally dissolved in the acrylic acid when acrylic acid is stored and/or transported under an atmosphere having 5% to 21% by volume of oxygen. In the desired polymerization of acrylic acid the content of dissolved oxygen in the acrylic acid is decreased, thus reducing the efficiency of MEHQ as a polymerization inhibitor enough for acrylic acid to undergo polymerization in the presence of MEHQ.

In addition to use as a polymerization inhibitor for the storage and/or transport of acrylic acid, MEHQ is also used as a polymerization inhibitor for the storage and/or transport of methacrylic acid, acrylic esters and/or methacrylic esters or of mixtures comprising one or more of said compounds.

Its widespread use makes MEHQ one of the most important storage and/or transport stabilizers for ethylenically unsaturated compounds, especially for acrylic acid, methacrylic acid, acrylic esters and methacrylic esters.

Ethylenically unsaturated compounds are stored in permanently installed vessels that are suitable for the purpose, such as storage tanks (see for example Acrylic Acid, A Summary of Safety and Handling, 4th edition 2013, 7 Bulk Storage Facilities and Accessories). Preferably, the storage of the ethylenically unsaturated compounds in the respective vessels is preferably for at least one hour, more preferably at least 10 hours, most preferably at least 100 hours. Although the duration of storage under appropriate conditions is theoretically unlimited, the duration of storage is generally reduced to a minimum for economic reasons. Preferably, the storage of the ethylenically unsaturated compounds in the respective vessels is for not more than 180 days, more preferably for not more than 90 days, most preferably for not more than 30 days. Particularly preferred ranges result from the free combination of the aforementioned lower and upper ranges.

Ethylenically unsaturated compounds are typically transported in suitable transportable vessels, such as tanks or drums, by ship, rail and/or truck (see for example Acrylic Acid, A Summary of Safety and Handling, 4th edition 2013, 9 Safe Transport of Acrylic Acid). The transportable vessels may of course also be permanently installed on the corresponding means of transportation, for example tanker ships or rail tank cars. It is of course also possible for vessels to be stored for a certain time in a particular place before they are transported to another site. Ethylenically unsaturated compounds may also be transported in pipelines or hoses, for example to a storage tank after purification of the ethylenically unsaturated compound, in the case of transfer from the storage tank into a transportable vessel, and/or in the case of transfer from one transportable vessel into another. Preferably, the transportation of the ethylenically unsaturated compounds in the respective vessels is preferably for at least one hour, more preferably at least 10 hours, most preferably at least 100 hours. Although the duration under appropriate conditions is theoretically unlimited, the duration is generally reduced to a minimum for economic and safety reasons. Preferably, the transport of the ethylenically unsaturated compounds in the respective vessels is for not more than 180 days, more preferably for not more than 90 days, most preferably for not more than 30 days. Particularly preferred ranges result from the free combination of the aforementioned lower and upper ranges.

EP 0 398 633 A1 discloses a method of inhibiting polymerization of acidic monomers with sources of manganese and of phenylenediamines comprising NH groups.

JP 5140027 discloses the use of mixtures of a) phenols, such as phenol, hydroquinone, p-methoxyphenol, cresol and catechol, b) manganese salts and c) sterically hindered phenols, such as 2,5-di-tert-butylhydroquinone, 2,6-di-tert-butylphenol, 6-tert-butyl-m-cresol; 2,6-di-tert-butyl-p-cresol, 4-tert-butylcatechol, 2,4-dimethyl-6-tert-butylphenol, 2-tert-butylhydroquinone and 2-tert-butyl-4-methoxyphenol, for stabilization of acrylic acid, methacrylic acid and esters thereof.

U.S. Pat. No. 6,207,859 discloses a method of storage and transport of α,β-unsaturated carboxylic acids. The α,β-unsaturated carboxylic acids are stabilized against unwanted free-radical polymerization with a phenol and an at least divalent metal cation. The at least divalent metal cation is removed again after storage and transport.

It was an object of the present invention to provide a method of storage and/or transport of ethylenically unsaturated carboxylic acids. The polymerization inhibitors are to ensure sufficient stabilization of the ethylenically unsaturated carboxylic acids against undesired free-radical polymerization. In the desired free-radical polymerization, however, there should be no need to separate the polymerization inhibitors from the mixture before the polymerization. The polymerization inhibitors are therefore not to act as polymerization chain-transfer agents and/or polymerization inhibitors in the desired free-radical polymerization. Furthermore, the polymerization inhibitors should not lead to unwanted discoloration in the polymerization.

The object is achieved by a method of storage and/or transport of an ethylenically unsaturated carboxylic acid, where the ethylenically unsaturated carboxylic acid has a purity of at least 90% by weight, the mass of stored and/or transported ethylenically unsaturated carboxylic acid is at least 100 kg and the ethylenically unsaturated carboxylic acid has a temperature during storage and/or transport of less than 50° C., wherein the ethylenically unsaturated carboxylic acid comprises from 0.000002% to 0.00005% by weight (20 to 500 ppb) of manganese(II) ions, based on the total amount of ethylenically unsaturated carboxylic acid.

The purity of the ethylenically unsaturated carboxylic acid is preferably at least 95% by weight, more preferably at least 98% by weight, most preferably at least 99% by weight.

The mass of stored and/or transported ethylenically unsaturated carboxylic acid is preferably at least 200 kg, more preferably at least 500 kg, most preferably at least 1000 kg. The mass of stored and/or transported ethylenically unsaturated carboxylic acid is typically less than 10 000 000 kg.

The ethylenically unsaturated carboxylic acid has a temperature during storage and/or transport of preferably less than 45° C., more preferably less than 40° C., most preferably less than 35° C. In the case of acrylic acid, the temperature during storage and/or transport should be at least 15° C.

The ethylenically unsaturated carboxylic acid preferably comprises less than 0.01% by weight (100 ppm), preferably less than 0.001% by weight (10 ppm), more preferably less than 0.0002% by weight (2 ppm), most preferably less than 0.0001% by weight (1 ppm), of phenols, based in each case on the total amount of ethylenically unsaturated carboxylic acid.

The ethylenically unsaturated carboxylic acid preferably comprises essentially no phenol and/or essentially no phenylenediamine.

The ethylenically unsaturated carboxylic acid preferably comprises from 0.000002% to 0.00005% by weight (20 to 500 ppb), preferably from 0.000005% to 0.000045% by weight (50 to 450 ppb), more preferably from 0.00001% to 0.00004% by weight (100 to 400 ppb), most preferably from 0.000015% to 0.000035% by weight (150 to 350 ppb), of manganese(II) ions, based in each case on the total amount of ethylenically unsaturated carboxylic acid.

The ethylenically unsaturated carboxylic acids are preferably mono-, di- or triethylenically unsaturated C₃- to C₈-carboxylic acids.

Particular preference is given to acrylic acid, methacrylic acid, dimethacrylic acid, ethacrylic acid, citraconic acid, methylenemalonic acid, crotonic acid, fumaric acid, mesaconic acid, itaconic acid and maleic acid, or any desired mixtures of two or more of the aforementioned ethylenically unsaturated carboxylic acids.

Most particular preference is given to acrylic acid and methacrylic acid.

The ethylenically unsaturated carboxylic acids are typically stored and/or transported in stainless steel vessels. Suitable stainless steels comprise 16.5% to 19.5% by weight of chromium and from 8.0% to 13.5% by weight of nickel.

The ethylenically unsaturated carboxylic acid is typically stored and/or transported under an oxygen-containing atmosphere.

The ethylenically unsaturated carboxylic acid is preferably stored in a vessel under an oxygen-containing atmosphere having an oxygen content of 5% to 10% by volume and the mixture in the vessel is regularly recirculated, for example by pumped recirculation of the entirety of the tank contents at least once a week. The relatively low oxygen content prevents the formation of ignitable gas mixtures in the vessel. The recirculation replaces consumed dissolved oxygen in the liquid ethylenically unsaturated carboxylic acid.

The present invention further provides a method of free-radical polymerization, wherein at least one of the above-described ethylenically unsaturated carboxylic acids is polymerized by means of a polymerization initiator, and for the use of one of the above-described ethylenically unsaturated carboxylic acids for free-radical polymerization by means of a polymerization initiator.

By the free-radical polymerization of the invention, for example, water-soluble and water-swellable polyacrylic acids and sodium salts thereof are obtainable.

Free-radical polymerization itself is common knowledge and is typically conducted in solution. Suitable polymerization initiators are all carboxylic acids that can break down to free radicals under the chosen reaction conditions, for example thermal initiators, redox initiators, photoinitiators. Suitable thermal initiators are peroxomono- and -disulfates, and peroxomono- and -diphosphates. Suitable redox initiators are sodium peroxodisulfate/ascorbic acid, hydrogen peroxide/ascorbic acid, sodium peroxodisulfate/sodium hypophosphite, sodium peroxodisulfate/sodium bisulfite and hydrogen peroxide/sodium bisulfite.

### EXAMPLES

### Inhibition of Polymerization Experiments

The ethylenically unsaturated carboxylic acid used was distilled twice to remove the hydroquinone monomethyl ether (MEHQ) polymerization inhibitor. The stated amount of the specified polymerization inhibitor was added in each case to the ethylenically unsaturated carboxylic acid obtained. MEHQ and manganese(II) acetate were used.

0.5 ml of each mixture was transferred to a 1.8 ml ampoule and stored at the specified temperature in an air circulation oven.

In each test series, in each case three ampoules were filled with each mixture and tested, with the average time for complete polymerization determined by visual examination.

TABLE 1
Mixtures comprising acrylic acid

| Polymerization | | | Time before |
|---|---|---|---|
| inhibitor | Amount | Temperature | polymerization |
| none*) | | 80° C. | 3.6 h |
| MEHQ*) | 0.002% by wt. | 80° C. | 11.1 h |
| MEHQ*) | 0.004% by wt. | 80° C. | 14.6 h |
| MEHQ*) | 0.006% by wt. | 80° C. | 21.6 h |
| Mn²⁺ | 0.000002% by wt. | 80° C. | 12.0 h |
| Mn²⁺ | 0.000005% by wt. | 80° C. | 19.8 h |
| Mn²⁺ | 0.00001% by wt. | 80° C. | 37.9 h |
| Mn²⁺ | 0.00002% by wt. | 80° C. | >46 h |

*)comparative

Manganese(II) ions, even in one thousandth of the concentration, have a comparable effect to MEHQ.

### Polymerization Experiments

A reaction vessel was initially charged under a nitrogen atmosphere with 450 g of water. The initially charged reaction mixture was heated to 95° C. with stirring. On attainment of the temperature of 95° C., three streams were metered in with stirring and while maintaining the temperature.
Stream 1: 500 g of acrylic acid, metered in over 5 h
Stream 2: 15 g of sodium hypophosphite in 35 g of deionized water, metered in over 4.75 h
Stream 3: 5 g of sodium peroxodisulfate in 66.4 g of deionized water, metered in over 5.25 h

After addition of the three streams, the reaction mixture was stirred at 95° C. for a further hour. The reaction mixture was then cooled to room temperature and 80 g of water was added.

The acrylic acid used in stream 1 was stabilized with MEHQ or manganese(II) acetate.

The polymers obtained were analyzed by GPC (calibration with Na-PAA standard, eluent: 0.01 mol/I phosphate buffer pH=7.4 in distilled water containing 0.01 M NaN₃).

TABLE 2
Polymerization of acrylic acid

| Polymeri- | | Number | Weight | Polydis- |
|---|---|---|---|---|
| zation | | average | average | persity |
| inhibitor | Amount | Mn | Mw | Mw/Mn |
| MEHQ*) | 0.020% by wt. | 3240 g/mol | 11 100 g/mol | 3.4 |
| Mn²⁺ | 0.0000001% by wt. | 3550 g/mol | 11 200 g/mol | 3.2 |
| Mn²⁺ | 0.000002% by wt. | 3620 g/mol | 11 600 g/mol | 3.2 |
| Mn²⁺ | 0.000005% by wt. | 3170 g/mol | 10 500 g/mol | 3.3 |
| Mn²⁺ | 0.00001% by wt. | 3450 g/mol | 12 100 g/mol | 3.5 |
| Mn²⁺ | 0.00002% by wt. | 3510 g/mol | 10 600 g/mol | 3.0 |
| Mn²⁺ | 0.00004% by wt. | 3510 g/mol | 12 100 g/mol | 3.4 |

*)comparative

The manganese(II) ions with the concentrations examined lead to polyacrylic acids having a comparable molar mass to that when the standard concentration of MEHQ is used.

### Polymerization of Sodium Acrylate or Sodium Methacrylate

A reaction vessel was initially charged under a nitrogen atmosphere with 52 g of deionized water and 60 g of aqueous sodium acrylate solution or aqueous sodium methacrylate solution (in each case 37.2% strength by weight and stabilized with 0.01% by weight of MeHQ or 0.0000057% by weight of manganese(II) acetate). Then 0.4 g of sodium peroxodisulfate dissolved in 10 g of deionized water was added and the mixture was heated to 80° C. while stirring. The reaction mixture was stirred at 80° C. for 4 h and then cooled to ambient temperature.

In all experiments with MEHQ, the solution became discolored during heating (at about 60° C.).

1.-15. (canceled)

16. A method of storage and/or transport of an ethylenically unsaturated carboxylic acid, where the ethylenically unsaturated carboxylic acid has a purity of at least 90% by weight, the mass of stored and/or transported ethylenically unsaturated carboxylic acid is at least 100 kg and the ethylenically unsaturated carboxylic acid has a temperature during storage and/or transport of less than 50° C., wherein the ethylenically unsaturated carboxylic acid comprises from 0.000002% to 0.00005% by weight of manganese(II) ions, based on the total amount of ethylenically unsaturated carboxylic acid.

17. The method according to claim 16, wherein the ethylenically unsaturated carboxylic acid comprises less than 0.0001% by weight of phenols, based on the total amount of ethylenically unsaturated carboxylic acid.

18. The method according to claim 16, wherein the ethylenically unsaturated carboxylic acid comprises essentially no phenol and/or essentially no phenylenediamine.

19. The method according to claim 16, wherein the ethylenically unsaturated carboxylic acid comprises from 0.000005% to 0.000045% by weight of manganese(II) ions, based on the total amount of ethylenically unsaturated carboxylic acid.

20. The method according to claim 16, wherein the ethylenically unsaturated carboxylic acid comprises from 0.00001% to 0.00004% by weight of manganese(II) ions, based on the total amount of ethylenically unsaturated carboxylic acid.

21. The method according to claim 16, wherein the ethylenically unsaturated carboxylic acid comprises from 0.000015% to 0.000035% by weight of manganese(II) ions, based on the total amount of ethylenically unsaturated carboxylic acid.

22. The method according to claim 16, wherein storage and/or transport of the ethylenically unsaturated carboxylic acid extends over a period of at least 10 hours.

23. The method according to claim 16, wherein the ethylenically unsaturated carboxylic acid is acrylic acid and/or methacrylic acid.

24. The method according to claim 16, wherein the ethylenically unsaturated carboxylic acid is stored and/or transported in a steel vessel and the steel comprises from 16.5% to 19.5% by weight of chromium and from 8.0% to 13.5% by weight of nickel.

25. The method according to claim 16, where the ethylenically unsaturated carboxylic acid is stored and/or transported under an oxygen-containing atmosphere.

26. The method according to claim 16, where the ethylenically unsaturated carboxylic acid is stored in a vessel under an oxygen-containing atmosphere having an oxygen content of 5% to 10% by volume and the ethylenically unsaturated carboxylic acid in the vessel is regularly recirculated.

27. A method of free-radical polymerization, where at least one ethylenically unsaturated carboxylic acid is polymerized by means of a polymerization initiator, wherein the ethylenically unsaturated carboxylic acid used comprises from 0.000002% to 0.00005% by weight of manganese(II) ions, based on the total amount of ethylenically unsaturated carboxylic acid.

28. The method according to claim 27, wherein the ethylenically unsaturated carboxylic acid comprises from 0.000015% to 0.000035% by weight of manganese(II) ions, based on the total amount of ethylenically unsaturated carboxylic acid.

29. The use of an ethylenically unsaturated carboxylic acid for free-radical polymerization by means of a polymerization initiator, wherein the ethylenically unsaturated carboxylic acid used comprises from 0.000002% to 0.00005% by weight of manganese(II) ions, based on the total amount of ethylenically unsaturated carboxylic acid.

30. The use according to claim 29, wherein the ethylenically unsaturated carboxylic acid comprises from 0.000015% to 0.000035% by weight of manganese(II) ions, based on the total amount of ethylenically unsaturated carboxylic acid.
